(19) United States
(12) Patent Application Publication
(10) Pub. No.: US 2025/0299779 A1
(43) Pub. Date: Sep. 25, 2025

(54) DEEP LEARNING-BASED SYSTEM FOR RAPID AND ACCURATE BACTERIAL CLASSIFICATION

(71) Applicant: Galacto Corp, Stillwater, OK (US)

(72) Inventor: Daniel J. Mueller, Stillwater, OK (US)

(73) Assignee: Galacto Corp, Stillwater, OK (US)

(21) Appl. No.: 19/089,930

(22) Filed: Mar. 25, 2025

Related U.S. Application Data
(60) Provisional application No. 63/569,444, filed on Mar. 25, 2024.

(51) Int. Cl. G16B 40/00; G06N 3/045; G16B 50/30

(52) U.S. Cl. CPC G16B 40/00; G06N 3/045; G16B 50/30

(57) ABSTRACT

This disclosure is various methods and systems that utilize deep learning, specifically convolutional neural networks and recurrent neural networks to enable bacterial identification and classification by analyzing raw genomic sequences, such as the 16S rRNA gene and other preserved regions. The system involves multiple convolutional layers to extract and generalize features, correlate their presence, and ultimately classify the sequences into genera or species. RNNs, such as LSTMs, are used when the order of features matters, particularly in cases with padded regions or separators between gene segments.

### CROSS-REFERENCE TO RELATED APPLICATION

This application claims priority to U.S. Provisional Application Ser. No. 63/569,444, filed on Mar. 25, 2024, which application is incorporated by reference herein in its entirety.

### TECHNICAL FIELD

The present invention relates to the fields of microbiology, bioinformatics, and machine learning. Specifically, the invention provides various embodiments of a system and method for the automated identification and classification of bacteria using deep learning algorithms and genomic sequence data.

### BACKGROUND

Traditional bacterial identification methods such as culture-based techniques, biochemical assays, basic local alignment search tool (“BLAST”), and molecular methods like polymerase chain reaction (“PCR”) and targeted sequencing, are often time-consuming, labor-intensive, resource-demanding, and may have limited ability to classify novel or closely related strains.

There is a significant need for faster, more accurate, and automated methods to identify and classify bacteria in various applications, including clinical diagnostics, environmental monitoring, epidemiological investigations, and basic research, as well as existing and novel pathogenic detection and rapid identification for disaster/terror response.

### SUMMARY

One embodiment of the present invention is a classifier system, comprising the following components: (1) a processing component; (2) a memory comprising a non-transitory processor-readable medium storing processor-executable instructions; and (3) a classifier convolutional neural network that, when executed by the processing component, causes the processing component to perform the following steps. This system processes a 16S rRNA gene or other similarly preserved regions of a specific genomic sequence to a plurality of known and isolated samples of genomic sequences, by at least one of filtering, padding, similarity processing, detection or aggregation, to create an aggregate set and feeding the aggregate set directly into a neural network which is configured to classify the aggregate set into at least one known genera and at least one known species by employing two algorithms, with each of the two algorithms containing an ensemble network configuration.

Another embodiment of the present invention is a classification method implemented by a processing component and a non-transitory computer-readable recording medium storing instructions, wherein the processing component is configured to run an application. This embodiment of a method comprises the following steps: (1) extracting preserved regions of a plurality of genomic sequences with each sequence having a known genus and a known species from the database; (2) preprocessing, by the processing component, the extracted regions into preprocessed regions; (3) organizing, by the processing component, each of the preprocessed regions into corresponding data files which contain the regions from processing with labels associated for the genus and species to which each region corresponds; and (4) training at least one of a genus classifier model using the genus data files and a species classifier model using the species data files.

### BRIEF DESCRIPTION OF DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function. In the drawings:

FIG. 1 is a schematic representation of one embodiment of a classification system according to the present invention;

FIG. 2 is a screenshot of one embodiment of a user interface showing possible inputs and outputs;

FIG. 3 is one embodiment of a screenshot of a new file dialog to receive new file dialog input(s);

FIG. 4 is an example of one embodiment of screenshot output confidences demonstrating the nature of the output parameters;

FIG. 5 is a representation of one embodiment of a model architecture having numerous convolutional layers;

FIG. 6 is a detailed view of one embodiment of a structure of the direct input given to a CNN for feature extraction and processing, which demonstrates the use of ‘x’ and ‘p’ for separators and end padding respectively;

FIG. 7 is a diagram of an exemplary embodiment of one-hot encoding of a genomic sequence constructed in accordance with the present disclosure;

FIG. 8 is a diagram of an exemplary embodiment of a classifier output constructed in accordance with the present disclosure;

FIG. 9 is a diagram of an exemplary embodiment of a flanking scan process constructed in accordance with the present invention;

FIG. 10 shows one equation to calculate total permutations according to one embodiment of the present invention; and

FIGS. 11A and 11B are flowcharts of one embodiment each of a model training and prediction functions of the present invention, respectively.

### DETAILED DESCRIPTION

Before explaining at least one embodiment of the disclosure in detail, it is to be understood that the disclosure is not limited in its application to the details of construction, experiments, exemplary data, and/or the arrangement of the components set forth in the following description or illustrated in the drawings unless otherwise noted. The disclosure is capable of other embodiments or of being practiced or carried out in various ways. Also, it is to be understood that the phraseology and terminology employed herein is for purposes of description and should not be regarded as limiting.

As used in the description herein, the terms “comprises,” “comprising,” “includes,” “including,” “has,” “having,” or any other variations thereof, are intended to cover a nonexclusive inclusion. For example, unless otherwise noted, a process, method, article, or apparatus that comprises a list of elements is not necessarily limited to only those elements but may also include other elements not expressly listed or inherent to such process, method, article, or apparatus.

Further, unless expressly stated to the contrary, “or” refers to an inclusive and not to an exclusive “or”. For example, a condition A or B is satisfied by one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the inventive concept. This description should be read to include one or more, and the singular also includes the plural unless it is obvious that itis meant otherwise. Further, use of the term “plurality” is meant to convey “more than one” unless expressly stated to the contrary.

As used herein, qualifiers like “substantially,” “about,” “approximately,” and combinations and variations thereof, are intended to include not only the exact amount or value that they qualify, but also some slight deviations therefrom, which may be due to computing tolerances, computing error, manufacturing tolerances, measurement error, wear and tear, stresses exerted on various parts, and combinations thereof, for example.

As used herein, any reference to “one embodiment,” “an embodiment,” “some embodiments,” “one example,” “for example,” or “an example” means that a particular element, feature, structure, or characteristic described in connection with the embodiment is included in at least one embodiment and may be used in conjunction with other embodiments. The appearance of the phrase “in some embodiments” or “one example” in various places in the specification is not necessarily all referring to the same embodiment, for example.

The use of ordinal number terminology (i.e., “first”, “second”, “third”, “fourth”, etc.) is solely for the purpose of differentiating between two or more items and, unless explicitly stated otherwise, is not meant to imply any sequence or order of importance to one item over another.

The use of the term “at least one” or “one or more” will be understood to include one as well as any quantity more than one. In addition, the use of the phrase “at least one of X, Y, and Z” will be understood to include X alone, Y alone, and Z alone, as well as any combination of X, Y, and Z.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value. It is not intended that the scope of the invention be limited to the specific values recited when defining a range. All ranges are inclusive and combinable.

Circuitry, as needed herein to connect components (as will be known to one skilled in the art), may be analog and/or digital components, or one or more suitably programmed processors (e.g., microprocessors) and associated hardware and software, or hardwired logic. The term “processor” as used herein means a single processor or multiple processors working independently or together to collectively perform a task or a functional unit that interprets and executes instruction data. Also, “components” may perform one or more functions. The term “processing component,” refers to a central processing unit that can include hardware, such as a processor (e.g., microprocessor), an application specific integrated circuit (“ASIC”), a field programmable gate array (“FPGA”), a combination of hardware and software, software, and/or the like. A processing component comprises the hardware and software configured to perform or execute the models, methods, and process of the present invention including performing systematic operations upon data or information exemplified by functions such as data or information transferring, merging, sorting, and computing (e.g., arithmetic operations or logical operations).

Software may include one or more computer readable instruction that when executed by one or more component, e.g., a processor, causes the component to perform a specified function. It should be understood that the algorithms described herein may be stored on one or more non-transitory computer-readable medium. Exemplary non-transitory computer-readable media may include a non-volatile memory, a random access memory (“RAM”), a read only memory (“ROM”), a CD-ROM, a hard drive, a solid-state drive, a flash drive, a memory card, a DVD-ROM, a Blu-ray Disk, a laser disk, a magnetic disk, an optical drive, combinations thereof, and/or the like. Such non-transitory computer-readable media may be electrically based, optically based, magnetically based, resistive based, and/or the like.

As used herein, the terms “network-based,” “cloud-based,” and any variations thereof, are intended to include the provision of configurable computational resources on demand via interfacing with a computer and/or computer network, with software and/or data at least partially located on a computer and/or computer network.

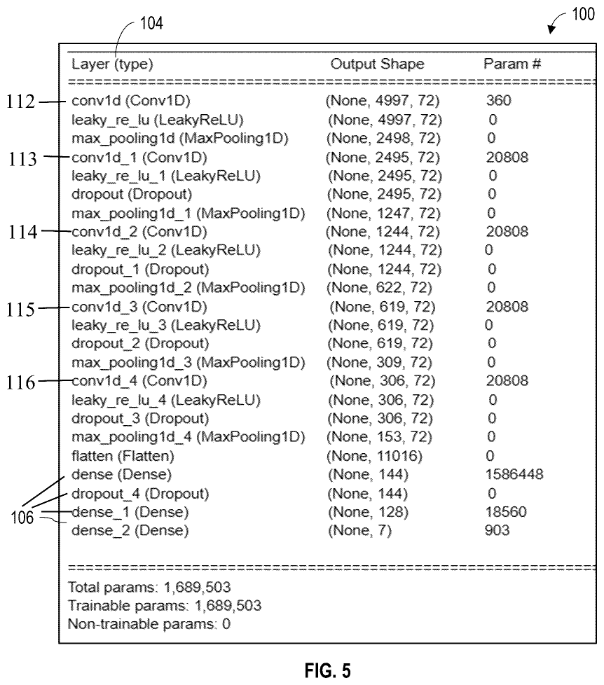

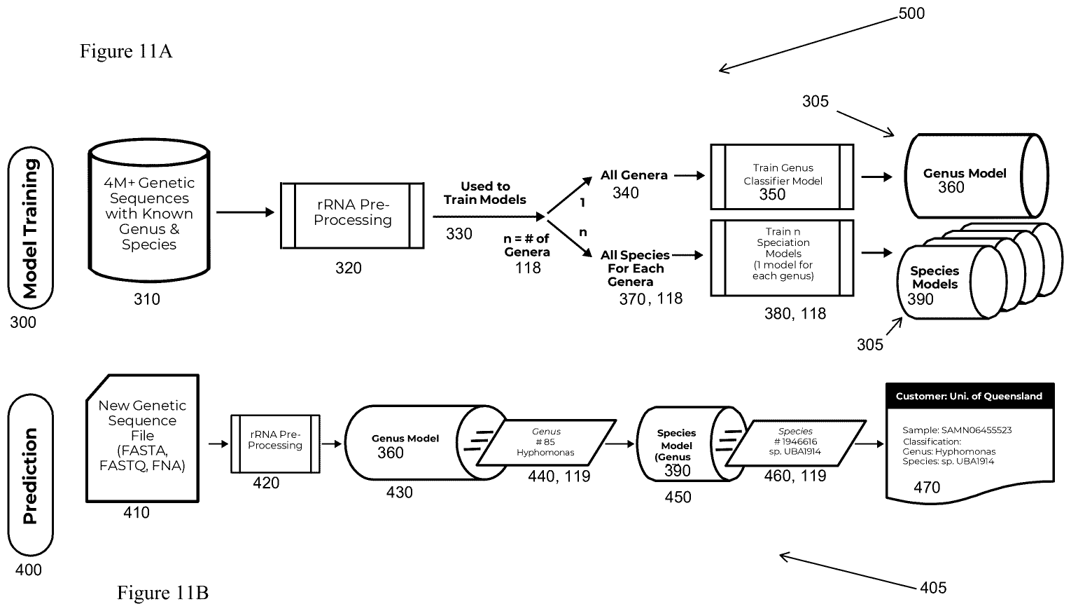

The present disclosure encompasses various embodiments of a classification system 10 and a classification method 500 that utilize the power of deep learning, specifically convolutional neural networks 100 (“CNNs”), to revolutionize bacterial identification and classification. These embodiments involve numerous convolutional layers 104 which aim to extract features from the genomic information of a preprocessed sample 58. The present invention does this in an orderly manner. First, in one embodiment, the invention sets a model equal to ‘Sequential 111’, after which a Conv1D layer 112 (a predetermined one-dimensional convolutional layer) is added to extract and generalize features that are present. The next layer, or second layer 113, is a Conv1D layer (another one-dimensional layer) designed to correlate the presence of multiple features present in the previous layer 112 to the next, or third, layer 114. Again, the first layer 112 is sequential. Then the output of that layer 112 is fed into a convolutional layer (the second layer 113) which is then fed into another convolutional layer (the third layer 114). After multiple or a plurality of these layers (unlimited number including but not limited to a fourth Conv1D layer 115 and a fifth Conv1D layer 116, see FIG. 5), there is an output layer 118 which contains the same number of classes as there are genera, or species per genera, depending on the type of classification model 305. In various embodiments of the present invention, the output layer 118 is the number of genera involved in training a model (see FIG. 11A at step 330 where n=# of genera and at the all species for each genera step 370 and train n specification models step 380). As further clarification of various examples of the present invention, the output layer 118 is a dense layer 106 that contains an equal number of nodes to that of the output classes available for the network (or CNN) to choose from. Each output layer 118 is given a score (for each parameter) from 0.0 to 1.0, where the highest scored value is typically chosen as the final output 440, 460. In many embodiments of the present invention, the output layer 118 is used to determine the ‘next-most-likely’ candidate of species. These output labels 119 (also referred to herein as the final output 440, 460 are the final genius and species determinations (see FIG. 11B) are what the model 305 is being trained to produce. In various embodiments of the present examples, the output labels 119 are the steps illustrated in FIG. 11B at 440 and 460, namely, the output 440 of the genus model 430 and the output 460 of the species model 450 when processing a new genetic sequence 410 through a prediction model 405 of the present invention. In one embodiment of the present invention, the user “feeds” the model with “known good” labels 310. Then when the model 305 encounters new data 58, it can arrive at these classification labels 470 (see FIGS. 11A and 11B). In the various embodiments of the present invention, “classes” refers to the number of nodes in the final layer, which corresponds to the cardinal number of genera or species within a genera, depending on the type of model being used. “Sequential”, as is known in the art (or field) and, as opposed to a non-sequential network such as a Recurrent Neural Network (RNN) or a Long-Short Term Memory (LSTM) Network, sets an artificial intelligence model to process data as a sequence or sequentially.

The various embodiments of classification methods 500 of the present invention incorporate a method of model training 300 to produce a trained model 305 that generates a genus model 360 and species models 390 (see FIG. 11A) and method of predicting 400 the genus and species of an input 58 by employing a prediction model 405 (see FIG. 11B). The prediction model 405 employs the genus model 360 and the species model(s) 390 from the trained model 305.

Recurrent neural network(s) (“RNN(s)”), such as a long short-term memory (“LSTM”), can be used to classify genomic sequences as well, where the order of features present matters. This is useful in cases where the preprocessing introduces padded regions 128 or ‘x’ separators 130 between gene segments 124. The classification systems 10 and methods 500 of the present invention analyzes raw genomic sequences 410, such as the 16S rRNA gene (and any variation thereof such as 23S rRNA, 5S rRNA, 18S rRNA, DNA polymerases, helicases, topoisomerases, RecA, RNA polymerase subunits [rpoA, rpoB, rpoC], ribosomal proteins, elongation factors, aminoacyl-tRNA synthetases, actin, tubulin, myosin, histones, cyclins, cyclin-dependent kinases, kinases, phosphatases, GTPases, Homeobox [Hox] genes, and any other preserved regions which may or may not be identified by an algorithm based on a regional or other similarity search or similar process, etc.), to extract complex patterns and features that enable rapid and accurate identification at various taxonomic levels 470 (e.g., genus, species, as well as higher order taxonomic classification levels or arbitrary classifications).

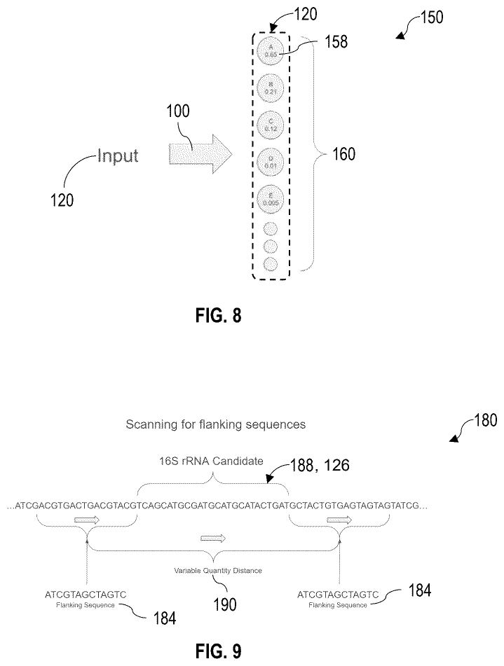

Generally, various embodiments of the classification system 10 and method 500 utilize deep learning, specifically convolutional neural networks 100 (“CNNs”), to enable bacterial identification and classification. The classification systems 10 and methods 400 involve numerous convolutional layers 104 (shown in FIG. 5) which aim to extract features from the genomic information of a preprocessed or full sample. First, in one embodiment, the classification system 10 sets a model 305 equal to ‘Sequential’, after which a Conv1D layer 112 (the first layer) is added to extract and generalize features present. The next layer (the second layer 113) is a Conv1D layer designed to correlate the presence of multiple features present in the previous, first layer 112 to the next layer (the third layer 114). After multiple of these layers, there is an output layer 118 which contains the same number of classes as there are genera, or species (FIG. 8). In some embodiments, RNNs such as an LSTM can be used to classify genomic sequences as well, where the order of features present matters. This is useful in cases where the preprocessing introduces padded regions or ‘x’ separators 130 between gene segments 124. Embodiments of the classification systems 10 and methods 500 analyze raw genomic sequences, such as the 16S rRNA gene (a nonlimiting example used herein) (and any variation thereof such as 23S rRNA, 5S rRNA, 18S rRNA, DNA polymerases, helicases, topoisomerases, RecA, RNA polymerase subunits [rpoA, rpoB, rpoC], ribosomal proteins, elongation factors, aminoacyl-tRNA synthetases, actin, tubulin, myosin, histones, cyclins, cyclin-dependent kinases, kinases, phosphatases, GTPases, Homeobox [Hox] genes, and any other preserved regions which may or may not be identified by an algorithm based on a regional or other similarity search or similar process, and/or the like), to extract complex patterns and features that enable rapid and accurate identification at various taxonomic levels (e.g., genus, species, as well as higher order taxonomic classification levels or arbitrary classifications).

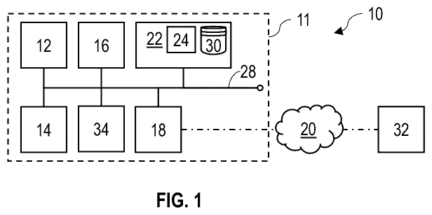

Referring now to the drawings, and in particular to FIG. 1, shown therein is a block diagram of an exemplary embodiment of a classification system 10 (which can be configured to perform the methods 400 of the present invention) constructed in accordance with the present disclosure. In some implementations, the classification system 10 may comprise a classification device 11 including one or more input device 12 (hereinafter “input device 12”), one or more output device 14 (hereinafter “output device 14”), one or more processing component 16 (hereinafter “processing component 16”), one or more communication device 18 (hereinafter “communication device 18”) capable of interfacing with a network 20, and one or more memory 22 (hereinafter “memory 22”) storing processor-executable code and/or application(s) 24 (hereinafter “application 24”).

As shown in FIG. 1, the input device 12, the output device 14, the processing component 16, the communication device 18, and the memory 22 may be connected via a path 28 such as a data bus that permits communication among the components of the classification device 11.

The input device 12 may be capable of receiving information input from a user and/or the processing component 16 and transmitting such information to other components of classification device 11, the classification system 10 and/or the network 20. The input device 12 may include, but is not limited to, implementation as a keyboard, a touchscreen, a mouse, a trackball, a microphone, a camera, a fingerprint reader, an infrared port, an optical port, a cell phone, a smart phone, a PDA, a remote control, a fax machine, a wearable communication device, a network interface, combinations thereof, and/or the like, for example.

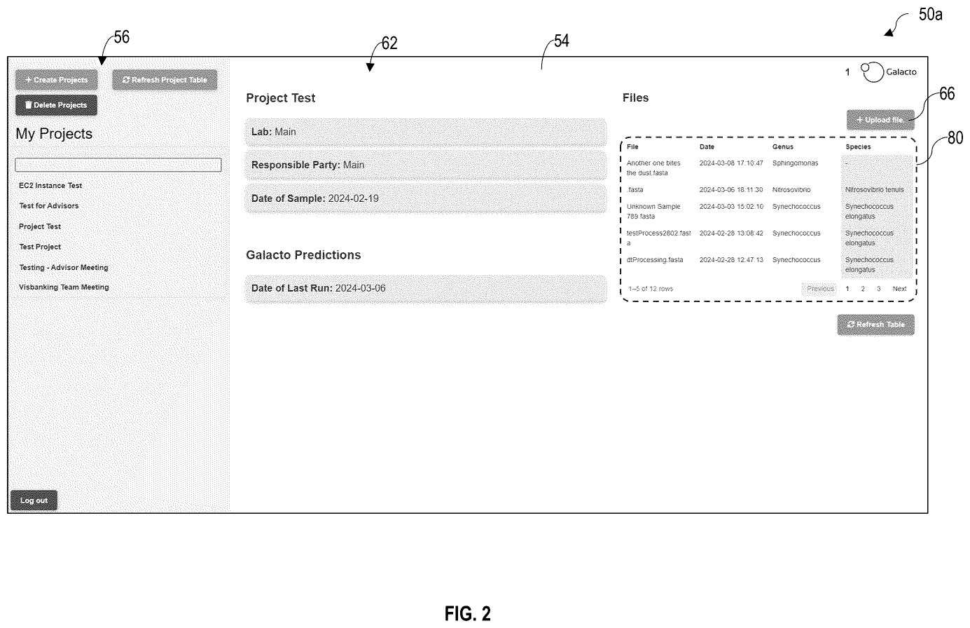

The output device 14 may be capable of outputting information in a form perceivable by the processing component 16. Implementations of the output device 14 may include, but are not limited to, a computer monitor, a screen, a touchscreen, a speaker, a website, a television set, a smart phone, a PDA, a cell phone, a fax machine, a printer, a laptop computer, a haptic feedback generator, an olfactory generator, combinations thereof, and the like, for example. It is to be understood that in some exemplary embodiments, the input device 12 and the output device 14 may be implemented as a single device, such as, for example, a touchscreen of a computer, a tablet, or a smartphone. It is to be further understood that as used herein the term user (e.g., the user) is not limited to a human being, and may comprise a computer, a server, a website, a processor, a network interface, a user terminal, a virtual computer, combinations thereof, and/or the like, for example. The output device 14 may display a user interface 54 (see FIG. 2).

The processing component 16 may be implemented as a single processor or multiple processors working together, or independently, to execute the application 24 as described herein. It is to be understood, that in certain embodiments using more than one processing component 16, the processing components 16 may be located remotely from one another, located in the same location, or comprising a unitary multi-core processor, or a combination thereof. The processing component 16 may be capable of reading and/or executing processor-executable code and/or capable of creating, manipulating, retrieving, altering, and/or storing data structures into the memory 22 such as in a database 30. The processing component 16 may be capable of communicating with the memory 22 via the path 28 (e.g., the data bus). The processing component 16 may be capable of communicating with the input device 12 and/or the output device 14 communicably coupled, or otherwise connected, to the classification device 11 of the classification system 10.

The processing component 16 may be further capable of interfacing and/or communicating with a server system 32 via the network 20 using the communication device 18. For example, the processing component 16 may be capable of communicating via the network 20 by exchanging signals (e.g., analog, digital, optical, and/or the like) via one or more port (e.g., physical ports or virtual ports) using a network protocol to provide updated information to the application 24 or the user interface 54. In one embodiment, the server system 32 is another embodiment of the classification device 11, however, the server system 32 may be constructed, for example, as one or more server having a plurality of CPUs, GPUs, NPUs, TPUs, and/or the like, or a combination thereof. The server system 32 may thus have a processing power available to both execute, or run, an AI model (e.g., the classifier CNN 100), as well as train, fine-tune, pre-train, instruction-tune, and/or align the AI model. The server system 32 may be specially designed to handle large-scale datasets efficiently. The classification system 10 (and the server system 32) can support diverse applications in research, industry, government use, and the like. The server system 32 enables said support through a neural network which minimizes the number of neurons necessary for classification purposes, which results in fewer computations leading to the end result. In this way, the technical problem of requiring significant computing resources is overcome by the present disclosure including the classification system 10.

In one implementation, the processing component 16 may be operable to receive the electrical signals from an artificial intelligence (“AI”) processor 34. The AI processor 34 may be constructed in accordance with the processing component 16, for example, and, in some embodiments, may be incorporated into the processing component 16. In some embodiments, the AI processor 34 may be separate from the processing component 16 but may work together with the processing component 16 to execute the application 24 and/or access the memory 22. In one embodiment, the AI processor 34 may operate at the request of, or be instructed to execute code by, the processing component 16.

Exemplary implementations of the processing component 16 may include, but are not limited to, a digital signal processor (“DSP”), a central processing unit (“CPU”), a graphical processing unit (“GPU”), a neural processing unit (“NPU”), a tensor processing unit (“TPU”), a field programmable gate array (“FPGA”), a microprocessor, a multi-core processor, an application specific integrated circuit (“ASIC”), combinations thereof, and/or the like, for example. The processing component 16 may include one or more processing component 16, having the same or different implementations, working together, or independently, and located locally, or remotely, e.g., accessible via the network 20 such as located in the server system 32, and may include a multi-core, multi-processor component. As such, the application 24 may be considered a cloud-based application 24, enabling access to powerful computing resources of the server system 32 and simplifying user experience via the user interface 54. This implementation as a cloud-based application 24 also drastically reduces processing time, as CUDA and tensor cores (e.g., the AI processors 34) allow the processing component 16 to perform matrix multiplication at much faster rates.

In one implementation, the memory 22 may be one or more non-transitory processor-readable medium. The memory 22 may store processor-executable instructions, such as the application 24, that, when executed by the processing component 16, causes the processing component 16 of the classification device 11 to perform an action such as communicate with or control one or more component of the classification device 11 and the classification system 10 and/or to perform one or more process such as the classification system 10. The memory 22 may be one or more memory 22 working together, or independently, to store processor-executable code and may be located locally or remotely, e.g., accessible via the network 20.

In some implementations, the memory 22 may be located in the same physical location as the classification device 11, and/or one or more memory 22 may be located remotely from the classification device 11 such as in the server system 32. For example, the memory 22 may be located remotely from the classification device 11 and communicate with the processing component 16 via the network 20. Additionally, when more than one memory 22 is used, a first memory may be located in the same physical location as the processing component 16, and additional memory may be located in a location physically remote from the processing component 16. Additionally, the memory 22 may be implemented as a “cloud” non-transitory processor-readable medium (i.e., the one or more memory 22 may be partially or completely based on or accessed using the network 20).

The memory 22 may store processor-executable code and/or information comprising the database 30 and the application 24. In some embodiments, the application 24 may be stored as a compiled application file, such as an executable file, for example, or in a structure (or unstructured) format, such as, e.g., in a non-compiled file. The application 24 may be stored in a computer-readable format, and may, in some embodiments, further be stored in a human-readable format.

In some implementations, the database 30 may be a time-series database, a relational database, a vector database, or a non-relational database. Examples of such databases include DB2®, Microsoft® Access, Microsoft® SQL Server, Oracle®, mySQL, PostgreSQL, MongoDB, Apache Cassandra, InfluxDB, Prometheus, Redis, Elasticsearch, TimescaleDB, Chroma, Pinecone, Weaviate, and/or the like. It should be understood that these examples have been provided for the purposes of illustration only and should not be construed as limiting the presently disclosed inventive concepts. The database 30 may be centralized or distributed across multiple systems.

In one embodiment, the database 30 may be a centralized database with a distributed backup database, a distributed database with a centralized backup database, a distributed database with a distributed backup database, or a centralized database with a centralized backup database. In one embodiment, the database 30 abides by, or exceeds, the 3-2-1 backup best practices. In one embodiment, each backup database is maintained as a real-time backup database, e.g., the backup database may be a mirror of the database 30.

In some implementations, the classification device 11 may include, but is not limited to, implementations as a personal computer, a cellular telephone, a smart phone, a network-capable television set, a tablet, a laptop computer, a desktop computer, a network-capable handheld device, a server, a digital video recorder, a wearable network-capable device, a virtual reality/augmented reality device, and/or the like.

In one implementation, the network 20 may permit bi-directional communication of information and/or data between the server system 32 and/or the classification device 11 of the classification system 10. The network 20 may interface with the classification device 11 and/or the server system 32 in a variety of ways. For example, in some embodiments, the network 20 may interface by optical and/or electronic interfaces, and/or may use a plurality of network topographies and/or protocols including, but not limited to, Ethernet, TCP/IP, circuit switched path, combinations thereof, and/or the like, as described above.

In some embodiments, the network 20 may be the Internet and/or other network. For example, if the network 20 is the Internet, the classification device 11 may interact with the server system 32 via the user interface 54 implemented on the output device 14 and/or the input device 12, such as a series of web pages or private internal web pages of a company or corporation, which may be written in hypertext markup language (HTML/PHP) and may utilize one or more suitable framework (such as JavaScript, Python, Flask, Django, and/or the like), for example. It should be noted that the user interface 54 of the classification device 11 may be another type of interface including, but not limited to, a Windows®-based application, a tablet-based application, a mobile web interface, an application running on a mobile device, a virtual-reality interface, an augmented-reality interface, and/or the like.

The network 20 may be almost any type of network. For example, in some embodiments, the network 20 may be a version of an Internet network (e.g., exist in a TCP/IP-based network). In one embodiment, the network 20 is the Internet. It should be noted, however, that the network 20 may be almost any type of wireless network and may be implemented as the World Wide Web (or Internet), a local area network (“LAN”), a wide area network (“WAN”), a low power wide area network “LPWAN”, a LoRa network (e.g., “LoRaWAN”), a metropolitan network, a wireless network, wireless networking technology a “WiFi network”, a cellular network, a Bluetooth network, a Global System for Mobile Communications (“GSM”) network, a code division multiple access (“CDMA”) network, a 3G network, a 4G network, a long term evolution (“LTE”) network, a 5G network, a satellite network, a radio network, an optical network, a shortwave wireless network, a long-wave wireless network, combinations thereof, and/or the like. It is conceivable that in the near future, embodiments of the present disclosure may use more advanced networking topologies.

The number of devices illustrated in FIG. 1 is provided for explanatory purposes. In practice, there may be additional devices, fewer devices, different devices, or differently arranged devices than are shown in FIG. 1. Furthermore, two or more of the devices illustrated in FIG. 1 may be implemented within a single device, or a single device illustrated in FIG. 1 may be implemented as multiple, distributed devices. Additionally, or alternatively, one or more of the devices of the classification system 10 may perform one or more functions described as being performed by another one or more of the devices of the classification system 10. Devices of the classification system 10 may interconnect via wired connections, wireless connections, or a combination thereof.

Various embodiments of the classification system 10 and method of the present invention significantly accelerate the classification (or identification) method 500, providing results in a fraction of the time compared to traditional methods. The classification system 10 and method 500 do this by automating the feature extraction process, thus eliminating the need for manual processing. The classification system 10 is able to correlate the bulk of the features present at the same time in the first layer, drawing correlations through the various layers to the output, meaning the user does not have to manually associate regions with various hereditary relationships to the output. The model architecture (shown in FIG. 5) selected is optimized to have a particular number of layers, with a high enough number of layers to capture complexities present in the genomic sequence, but not so many layers as to cause a lack of convergence.

Additionally, the various embodiments of a classification system 10 and methods 500 of the present invention outperform traditional methods in correctly classifying known biological organisms and biological organic structures and has the potential to identify novel strains more effectively. Various embodiments of the classification system 10 and methods 500 do this by associating all known or identifiable features at the same time in layer one, then further interrelating them in subsequent layers, followed by a direct correlation to output in the final layer as detailed below and shown in FIG. 5. Traditional methods include such as manual processing of samples through microscopy, BLAST, and the like introduce errors and misclassifications because said methods require subjective analysis of the user, whereas the classification system 10 uses an objective computational system described herein to provide analysis with pre-trained models such as a classifier CNN 100. In this way, the classification system 10 drastically reduces the time required for bacterial identification, enabling faster decision-making. Moreover, the classification system 10 leverages computational resources at both the server system 32 and the classification device 11 and minimizes a need for expensive laboratory consumables and reagents by allowing the user to perform analysis on a sequenced genome, thus reducing the need for manual identification through other means.

Referring now to FIGS. 1-4, in combination, shown therein are screenshots 50a-c of a user interface 54 provided by one embodiment of and application 24 in accordance with the present disclosure. The user interface 54 may be utilized by a user, for example, by researchers, clinicians, government workers, geneticists, bacteriologists, microbiologists, and/or the like. The user interface 54 may provide one or more input 58 such as one or more button operable to receive an input from the user by the processing component 16 (see FIG. 2). The processing component 16 may respond to the input 58, e.g., by executing one or more processor-executable instructions (see FIG. 2). For example, the one or more inputs 58 may include a “new project” button 66 which, upon selection by the user, may cause the processing component 16 to create a new project, and provide a project ID, for example, to the memory 22, e.g., in the database 30 (see FIG. 1). The project ID may further be associated with one or more project property, such as a project name, a lab name, a responsible party (e.g., user responsible for the project, which may, in some embodiments, default to the user that created the project), and one or more date, such as a creation date, and update date, a sample date, and the like. A list of projects 56 can be made available to the user.

In some embodiments, the one or more project properties may be display for the user on the user interface 54, such as via outputs 62 (see FIG. 2). In some embodiments, the user may select a particular output 62 to update or edit a value of the one or more project properties.

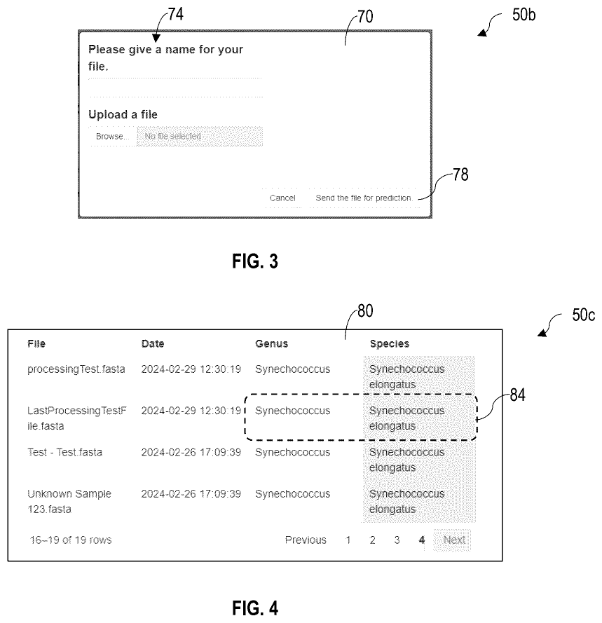

In one embodiment, upon selection of a “new file” input 66 by a user (FIG. 2), the processing component 16 may provide a new file dialog 70 (FIG. 3) operable to receive a new file input 58 from the user (or from a user device, or data stream) and to associate that new file 58 with the project ID, such as in the server system 32, memory 22 and/or the database 30 (FIG. 1). In one embodiment, the new file 58 is received by indicating a desire to input a new file 58 by clicking on a new file input 66 (which can be a button as shown in FIG. 2 or another appropriate input indicator) is received by utilizing the dialog 70 which may include one or more input 74 to receive one or more file properties from the user (see FIG. 3). For example, the one or more input 58 entered through a new file dialog box 74 (or other appropriate input mechanism) may include a file name and a file location (see FIG. 3). Upon selection of a confirmation button 78 (labeled as “Send the file for prediction” in the screenshot 50b), the processing component 16 may upload the new file located at the file location and save the new file, with the file name, to the memory 22, such as in the database 30, associated with the project ID (see FIG. 3.)

In one embodiment, upon uploading the new file (the input 58), the processing component 16 may execute a classification process 400 to cause the processing component 16 to classify the genomic sequence 58 stored in the new file as a particular bacteria species 470. For example, in one embodiment, the user interface 54 may further include an output portion 80 (also shown in FIG. 4) operable to display one or more result 84, 470 from the classification process 400. The one or more result 84 may include, for example, a genus and a species of the genomic sequence 58. In some embodiments, the results 84 may include additional classifications, for example, based on the phylogenetic tree. In this way, the processing component 16 presents classifier output 150 (FIG. 8) in a user-friendly format as the one or more result 84 in the output portion 80. Thus, the one or more result 84 may include a most probable genus and species, as well as potential alternative classifications, with respective confidence levels 158 (see FIG. 8) (alternatively, or additionally, including, an ‘unknown’ category for results that do not provide a high confidence, or that provide a confidence that does not meet a confidence threshold, e.g., 50%).

In one embodiment, the new file 58 may provide raw genomic data and may be in the form of FASTA or FASTQ file formats. In other embodiments, the one or more input 58 may receive a connection or stream linked to one or more of: one or more genomic repository, a user input, or from sequencing instruments (e.g., via an interface to the sequencing instrument, such as via an API). Throughout this disclosure, an input 58 includes and is alternatively referred to as a genomic sequence, a preprocessed sample, a new genome, an input genome, genomic data, a new sample, a new file, a new file input, an input (and variations thereon, collectively, input 58) and the new file input button 66 is a mechanism for entering an input 58 into the various systems 10 and methods 500 of the present invention.

In one embodiment, preserved regions of the input genome are used as biological markers to identify similarity with known classes of output (in genus, species, and/or the like format, or in arbitrary classifications of uniform and non-uniform type).

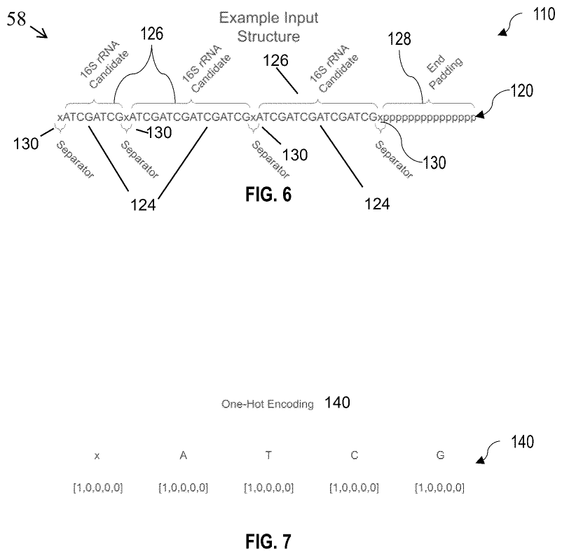

In one embodiment, the raw genomic data 58 may be either normalized via padding 128 to ensure consistency in input format and length (shown in FIG. 6). Alternatively, or additionally, the input layer (FIG. 5) may be modified in a neural translatory process to relate existing models down to smaller dimensionalities from a larger foundational model.

In one embodiment, the processing component 16 employs one-hot encoding (FIG. 7) to represent nucleotide sequences of the genomic sequence, in a suitable format for the classifier CNN 100 (FIG. 5). The processing component 16 may (alternatively or additionally) utilize other encoding methods, or direct processing via a transformer-based neural network or similar.

In one embodiment, the classification method 500 leverages the power of deep learning (machine learning) to achieve high levels of accuracy in identifying known bacterial species, as well as the potential to identify novel strains. The classification method 500 does this by recognizing the presence of features (e.g., through feature extraction), the interrelation of the extracted features, and then correlates the extracted features to the output, simultaneously.

In one embodiment, by automatically executing the classification method 500 upon upload of the new file 58, the classification system 10 minimizes a need for manual intervention of the user and lessens a need for the user to have a specialized expertise.

Referring now to FIG. 5, shown therein is a schematic representation of an exemplary embodiment of the classifier CNN 100 constructed in accordance with the present disclosure. The classifier CNN 100 is comprised of multiple neural network layers 104. As shown, a preprocessed genomic sequence 120 (FIG. 6) may be received as an input to the classifier CNN 100. Further, as shown, the neural network layers 104 as convolutional layers having one or more dense layer 106 provided for classification output. In the exemplary classifier CNN 100, a total of 1,689,503 parameters are provided for training, however, this number of parameters are provided for exemplary purposes and may include more or fewer trainable (or non-trainable) parameters. The number of trainable (or non-trainable) parameters may be selected based on a desirable accuracy, convergence, and/or available computing resources. The classifier CNN 100 may be a multi-layer CNN with convolutional layers, pooling layers, and fully connected layers as shown in FIG. 5. Layer configurations and hyperparameters may be optimized through experimentation. FIG. 5 shows five convolutional layers 112, 113, 114, 115, and 116, but variations on the systems 10 and methods 500 of the present invention can have different numbers of convolutional layers 104. As shown in FIG. 5 under the column labeled “Param #”, each of the rows that are not zero are their own layers. The first layer is Layer “conv1d (Conv1D)” 112; the second layer is Layer “conv1d_1” 113; the third layer is Layer “conv1d_2” 114, etc. Technically speaking, each row in the FIG. 5 table is a layer but the rows with a “Param #” that are non-zero are the beginning of each major layer. Finally, dense, dense_1 and dense_2 are also layers in the same way but possess a unique quality. A “dense” layer 106 is a fully connected layer, in other words, each node in the previous layer is connected to each node in the dense layer 106, just with different weights and biases.

As used herein, an “ensemble network configuration” refers to multiple CNNs 100. As a non-limiting example, for each genus classification, there are three distinctly-trained and structurally-identical models each of which provides its own predictions which are utilized to arrive at a final prediction which results from this ensemble network (the multiple models). These models are composed of the multiple layers of CNNs and dense layers that comprise an individual model. The ensemble network configuration includes utilization of a plurality of distinctly trained and structurally identical models which perform separate predictions to generate outputs, which as a whole, result in an output of higher accuracy on a validation set than each individual network alone.

In one embodiment, the classifier CNN 100 is (or has been) trained on a comprehensive and well-curated dataset of bacterial genomes encompassing a broad range of known genera and species 310, as well as viral, human genome, and other classifications which encompass all biological agents such as organic nanostructures and long-chaining organic molecules such as isolated genes, and/or the like (see FIG. 11A). Generally, the quality and diversity of the training dataset 310 are important for achieving high accuracy and generalization. The training dataset 310 may include a large and diverse dataset of bacterial genomes derived from publicly available repositories such as NCBI GenBank (U.S. National Institute of Health, Bethesda, Maryland) and others. The training dataset may be selected such to include datasets having quality and comprehensive genomic data. In this way, the classifier CNN 500 is optimized for analyzing genomic sequences. The classifier CNN 500 is trained on a vast and diverse dataset of bacterial genomes, enabling the classifier CNN 500 to learn intricate patterns associated with different species or organisms.

In one embodiment, the classifier CNN 500 may periodically be retrained on a new or updated training set. For example, continuous retraining and updating of the classifier CNN 500 may be beneficial to maintain performance of the classifier CNN 500 as new sequencing data becomes available, i.e., is introduced into the training dataset. Moreover, performance of the classifier CNN 100 may be periodically validated against standard reference models.

In one embodiment, the classifier CNN 500 may be trained (or retrained) on the server system 32. Moreover, the classification process utilizing the classifier CNN 500 may be performed on the server system 32 or on the classification device 11, such as by, or in conjunction with, the AI processor 34. As described here, references to actions performed by components of the classification device 11, such as to the processing component 16, may similarly be performed by a processing component of the server system 32. In some embodiments, training (or retraining) may be performed by the server system 32, while classification using the classifier CNN 500 may be performed by the classification device 11, such as a smart phone, tablet, laptop, desktop, and/or the like.

In one embodiment, preprocessed genomic sequences 120 (FIG. 6) are provided into the classifier CNN 100 where the processing component 16 executing the classifier CNN 500 extracts and analyzes features present in the genomic sequences, which include, but are not limited to: similarity, binary presence identification, and causal inference (such as measurement of an output chemical, product, or structure).

In one embodiment, the classifier CNN 500 can be provided as one or more local models for users for local inference (e.g., execution on the network 20 not being the Internet). The local models may be specialized and only include the genera of interest for a given experiment, with samples being processed further if the sample(s) contain a contaminate with a confidence score 158 low enough to register as not being contained by the dataset (e.g., below a contamination threshold). The processing component 16 may generate the local models via a convolutional neural network to provide a comprehensive manner of miniaturization of the classifier CNN 500 and may necessitate sending the local models in two (2) discrete portions.

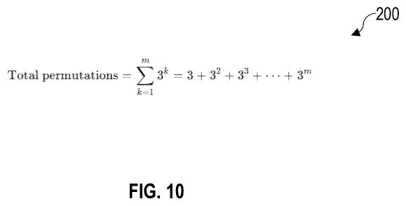

In one embodiment, the classification system 10 could incorporate additional AI models trained on specific groups of organisms or for particular applications (e.g., antibiotic resistance prediction, virulence profiling, etc.). These additional AI models may also be used as components of GANs (generative adversarial network), in the form of the discriminator (potentially with additional features added to the model architecture). A GAN contains a generator and a discriminator which tells real inputs apart from fake/generated inputs. The ability to process the presence of certain aspects of hereditary relationships, such as preserved motifs, with a neural network-based confidence value (and also in regard to the presence of other motifs associated), enables detection of hybrid species which are the combination of more than one species. Thus, if the confidence score 158 is below a certain threshold, as defined by a neural network, the neural network may classify the hybrid species as generated and provide the hybrid species a class label representing a most likely type of generated bacteria. This relationship is further represented below by the equation 200 in FIG. 10, where m is a number of species.

Referring now to FIG. 6, shown therein is a diagram of an exemplary embodiment of an input structure 110 of a preprocessed genomic sequence 120 constructed in accordance with the present disclosure. The preprocessed genomic sequence 120 may include one or more candidate regions 126 and may include padding 128, based on a difference between the genomic sequence and a required input length of the classification CNN 500. Each of the candidate regions 126 and the padding 128 may be separated by a separator 130. The separator 130 and the padding 128 may be, for example, a character or symbol that is not provided in any DNA/RNA sequence, such as an ‘x’ 130 for the separator 130 and a ‘p’ for the padding 128. The processing component 16 may retrieve the preprocessed genomic sequence 120 (from the memory 22 and/or from an output of a preprocessing function) and supply the preprocessed genomic sequence 120 to the classifier CNN 100. It should be understood that while the candidate regions 126 are shown in FIG. 6 to be a 16S rRNA candidate, the candidate regions 126 may be another genomic region(s) relevant for identification of the biological or organic sequence.

In one embodiment, the processing component 16 of the classification system 10 may generate the preprocessed genomic sequence 120 by executing one or more preprocessing function on the genomic sequence received from the new file dialog 70, for example, the processing component 16 may perform data cleaning, extraction, normalization, and one-hot encoding steps (shown in FIG. 7), and/or the like, to prepare the genomic sequences for analysis, e.g., to generate the preprocessed genomic sequence 120. In this way, the processing component 16 may enable quality control measured, sequence normalization, and one-hot encoding.

Referring now to FIG. 7, shown therein is a diagram of an exemplary embodiment of one-hot encoding of a genomic sequence 140 constructed in accordance with the present disclosure. Referring to FIG. 8, shown therein is a diagram of an exemplary embodiment of a classifier output 150 constructed in accordance with the present disclosure. The classifier output 150 may include a plurality of output parameters 154 (such as described in the one or more dense layer 106, FIG. 5). Each of the plurality of output parameters 154 may have a confidence score 158 (such as a percentage confident). The collection of the plurality of output parameters 154 having the confidence score 158 may be referred to as output confidences 160. In this way, based on learned or inferred patterns, the classifier CNN 500 assigns bacterial classifications with associated confidence scores 158 at various taxonomic or arbitrarily defined levels.

Referring now to FIG. 9, shown therein is a diagram of an exemplary embodiment of a flanking scan process 180 constructed in accordance with the present disclosure. The flanking scan process 180 may be performed by the processing component 16 to determine one or more flanking sequence 184 to determine a region of interest 188 (e.g., candidate region) having a variable distance 190.

FIGS. 11A and 11B illustrate the two primary components of one embodiment of the present invention classification method/model 500 as two flowcharts 300, 400. The flowchart in FIG. 11A illustrates the method by which training of some models occur 300 (or the creation of a trained model 305 comprising a genus model 360 and at least one species model 390). The flowchart in FIG. 11B demonstrates how the taxonomic classification of a previously unseen sample of genomic information 410 can be predicted 400 by preprocessing and classification using the trained models 360, 390 of the present invention.

The flowchart of FIG. 11A illustrates model training 300 of one embodiment of the present invention. Preprocessing 320 occurs whereby 16S (as a non-limiting example) or other similarly preserved regions are extracted from every genomic data file 310 being used to train the model(s) 330 of the present invention. The preprocessed files are organized by the known genera and species with which they are associated. Then, two types of models are trained using this data—Genera 360 and Species 390 models. For both models, the known genomic data collected 310 and preprocessed 320 with their associated genera 340 and species 370 labels are used to train a model 350, 380 for each genus and species.

To train these models, the preserved regions are fed into a network training algorithm by which the network learns to identify the genus or species through its preserved regions 350, 380. Following training, the resulting models 360, 390 are saved according to the labels for each genus and species and can be used by the prediction model 405 whose method 400 is illustrated in FIG. 11B.

In the second step (illustrated in the prediction flowchart 400 of FIG. 11B), after a similar rRNA (as a non-limiting example) preprocessing 420 that extracts the preserved regions of one genomic file 410, this extracted data is provided to a Genus model (step 430 employing genus model 360) which, in a rapid manner, outputs the predicted genus 440. Following this prediction 440, the species model (step 450 employing species model(s) 390) associated with that identified genus is then utilized to narrow the classification in the same manner. The extracted preserved region data is fed into the species model 450 which provides a prediction 460 of the species that this genomic data is associated with 470.

Another embodiment of the present invention is a method of converting an assembled genomic sequence into a classification, which utilizes any preserved regions. In the example used herein, bacteria all have 16S regions, so their presence can be used to (1) indicate that the sequence is a bacteria with a high degree of confidence, and (2) identify what the bacteria ‘is’ (genus & species). This method is useful because it relies on a small fraction of the computational steps normally required by the traditional method of alignment (see FIG. 11A).

Alignment is a traditional method in which a known genome is lined up with the identified candidate and a determination is made, nucleotide-wise, as to how similar they are. This requires millions of computations and increases exponentially with genome size since genomes are typically round and therefore have no defined “ends” to align. Another traditional method for evaluating DNA is employing search-space minimizer alignment techniques. “Search-space minimizer alignment techniques” or “search space minimizers” reduce the search space for DNA alignment by focusing on fixed-length sequences, rather than entire sequences. These methods improve the speed and efficiency of the analyses. Various embodiments of the present invention function as “search space minimizers” because they enable a user to reduce the amount or number of possible candidates (in some circumstances, to a few hundred) at which point traditional alignment could be used to produce a final validation, which produces the results that traditional labs are used to. The methods illustrated in FIGS. 11A and 11B and embodied in FIGS. 1-10 use a trained convolutional network (ensemble) to identify the preserved regions which allow for unique classification into taxonomic identifiers (e.g. genus, species). This method takes around 5 seconds, as opposed to what is often a 40+ minutes process for standard methods.

Another embodiment of the present invention is a classifier system that comprises the following components: (1) a processing component; and (2) a memory comprising a non-transitory processor-readable medium storing processor-executable instructions and a classifier convolutional neural network that, when executed by the processing component, causes the processing component to perform step of: (a) filtering and, padding, and 16S (as a non-limiting example) extraction or other similarly preserved region similarity to all known and isolated samples with detection and aggregation into an aggregate set; and (b) feeding the aggregate set directly into a neural network which is configured to classify the aggregate set into known genera and species in a series of two algorithms, which contain an ensemble network configuration. Another embodiment of the present invention builds upon this embodiment by configuring the ensemble network configuration to include utilization of multiple separate and distinguished algorithms which perform separate analyses to generate an output of higher accuracy on a validation set than each individual network alone. An ensemble network configuration is a standard terminology in machine learning algorithms, which refers to multiple networks which give a consensus on an output.

One embodiment of the present invention is a classifier system that comprises a processing component and a memory comprising a non-transitory processor-readable medium storing processor-executable instructions and a classifier convolutional neural network that, when executed by the processing component, causes the processing component to perform the following steps. The embodiment processes a 16S rRNA gene or other similarly preserved regions of a specific genomic sequence to a plurality of known and isolated samples of genomic sequences, by at least one of filtering, padding, similarity processing, detection or aggregation, to create an aggregate set and feeding the aggregate set directly into a neural network which is configured to classify the aggregate set into at least one known genera and at least one known species by employing two algorithms, wherein each of the two algorithms containing an ensemble network configuration. In some embodiments of a classifier system of this embodiment the ensemble network configuration includes utilization of a plurality of distinctly trained and structurally identical models configured to perform separate predictions to generate outputs, wherein the outputs of the models in the aggregate have a higher accuracy upon validation than each individual network alone.

Various embodiments of a classifier system of the present invention incorporate a convolutional neural network configured to extract features from a sample of genomic information through a system of convolutional layers. These embodiments comprise the following: setting a model equal to process data as a sequence, after which a first convolutional layer is added to extract and generalize features of the sample of genomic information; designing a second convolutional layer to correlate a presence of multiple features in the first convolutional layer to a third convolutional layer; and generating an output layer containing the same number of classes as there are genera or species.

Various embodiments of a classification system of the present invention are configured so that the processing component employs one-hot encoding to represent nucleotide sequences of the genomic sequences in a format configured for use with the classifier convolutional neural network.

Various embodiments of a classification system of the present invention is configured so that the classifier convolutional neural network is comprised of at least two convolutional layers and at least one dense layer. Various embodiments are configured such that the classifier convolutional neural network is trained on a dataset selected from the group consisting of bacterial genomes, human genomes, and viral genomes. Various embodiments are configured such that the classifier convolutional neural network extracts and analyzes features present in the genomic sequences, with the features are selected from the group consisting of similarity, binary presence identification and causal inference.

One embodiment of a classification method of the present invention is implemented by a processing component and a non-transitory computer-readable recording medium storing instructions, wherein the processing component is configured to run an application comprising the steps of: extracting preserved regions of a plurality of genomic sequences with each sequence having a known genus and a known species from the database; preprocessing, by the processing component, the extracted regions into preprocessed regions; organizing, by the processing component, each of the preprocessed regions into corresponding data files which contain the regions from processing with labels associated for the genus and species to which each region corresponds; and training at least one of a genus classifier model using the genus data files and a species classifier model using the species data files.

Various embodiments of a classification method of the present invention include preprocessed regions into a network training algorithm configured to identify a genus or a species of the genomic data to train the models, labeling the genus data files or species data files, and saving the trained models according to the provided labels of the genus and species data files. Various embodiments of a classification method of the present invention also comprise the following: receiving a genomic sequence file at the input device; preprocessing the genomic sequence file; analyzing the genomic sequence file with the genus classifier model to identify a corresponding genus output; and analyzing the genomic sequence file with the species classifier model associated with the genus output to identify a corresponding species output.

For some embodiments of the present invention classification method the genomic sequence file is in the form of FASTA or FASTQ file formats. For some embodiments of the classification method the preprocessed regions are used as biological markers to identify similarities with known classes of genus or species outputs. For some embodiments of the classification method the genomic sequence file is normalized by using padding to ensure consistency in input format and length. For some embodiments of the classification method the processing component comprises a central processing unit comprising at least one processor configured to perform systematic operations upon data.

From the above description, it is clear that the inventive concepts disclosed and claimed herein are well adapted to carry out the objects and to attain the advantages mentioned herein, as well as those inherent in the invention. While exemplary embodiments of the inventive concepts have been described for purposes of this disclosure, it will be understood that numerous changes may be made which will readily suggest themselves to those skilled in the art and which are accomplished within the spirit of the inventive concepts disclosed and claimed herein.

For example, the classification system 10 disclosed herein may be utilized for: Clinical Diagnostics (e.g., by enabling rapid and accurate bacterial identification for diagnosis of infectious diseases, leading to improved treatment decisions); Environmental Monitoring (e.g., by enabling identification of bacterial species in water, soil, and air samples for environmental health assessments); Pathogen Identification (e.g., by enabling identification of bacterial—and other biological—genera, species, or other category of label or classification from isolated samples); Microbiome Research (e.g., by enabling characterization of complex microbial communities in diverse ecosystems); Food Safety (e.g., by enabling monitoring for pathogenic bacteria in food products and production environments); and Industrial Microbiology (e.g., by enabling identification of bacterial strains for biotechnology and biopharmaceutical applications).

The foregoing description provides illustration and description, but is not intended to be exhaustive or to limit the inventive concepts to the precise form disclosed. Modifications and variations are possible in light of the above teachings or may be acquired from practice of the methodologies set forth in the present disclosure.

Even though particular combinations of features and steps are recited in the claims and/or disclosed in the specification, these combinations are not intended to limit the disclosure. In fact, many of these features and steps may be combined in ways not specifically recited in the claims and/or disclosed in the specification. Although each dependent claim listed below may directly depend on only one other claim, the disclosure includes each dependent claim in combination with every other claim in the claim set.

What is claimed is:

1. A classifier system, comprising:
a processing component; and
a memory comprising a non-transitory processor-readable medium storing processor-executable instructions and a classifier convolutional neural network that, when executed by the processing component, causes the processing component to perform the steps comprising:
processing a 16S rRNA gene or other similarly preserved regions of a specific genomic sequence to a plurality of known and isolated samples of genomic sequences, by at least one of filtering, padding, similarity processing, detection or aggregation, to create an aggregate set; and
feeding the aggregate set directly into a neural network which is configured to classify the aggregate set into at least one known genera and at least one known species by employing two algorithms, wherein each of the two algorithms containing an ensemble network configuration.

2. The classifier system of claim 1, wherein the ensemble network configuration includes utilization of a plurality of distinctly trained and structurally identical models configured to perform separate predictions to generate outputs, wherein the outputs of the models in the aggregate have a higher accuracy upon validation than each individual network alone.

3. The classifier system of claim 1, wherein the convolutional neural network extracts features from a sample of genomic information through a system of convolutional layers comprising:
setting a model equal to process data as a sequence, after which a first convolutional layer is added to extract and generalize features of the sample of genomic information;
designing a second convolutional layer to correlate a presence of multiple features in the first convolutional layer to a third convolutional layer; and
generating an output layer containing the same number of classes as there are genera or species.

4. The classification system of claim 1, wherein the processing component employs one-hot encoding to represent nucleotide sequences of the genomic sequences in a format configured for use with the classifier convolutional neural network.

5. The classification system of claim 1, wherein the classifier convolutional neural network is comprised of at least two convolutional layers and at least one dense layer.

6. The classification system of claim 1, wherein the classifier convolutional neural network is trained on a dataset selected from the group consisting of bacterial genomes, human genomes, and viral genomes.

7. The classification system of claim 1, wherein the classifier convolutional neural network extracts and analyzes features present in the genomic sequences, with the features are selected from the group consisting of similarity, binary presence identification and causal inference.

8. A classification method implemented by a processing component and a non-transitory computer-readable recording medium storing instructions, wherein the processing component is configured to run an application comprising the steps of:
extracting preserved regions of a plurality of genomic sequences with each sequence having a known genus and a known species from the database;
preprocessing, by the processing component, the extracted regions into preprocessed regions;
organizing, by the processing component, each of the preprocessed regions into corresponding data files which contain the regions from processing with labels associated for the genus and species to which each region corresponds; and
training at least one of a genus classifier model using the genus data files and a species classifier model using the species data files.

9. The classification method of claim 8, further comprising:
inputting the preprocessed regions into a network training algorithm configured to identify a genus or a species of the genomic data to train the models;
labeling the genus data files or species data files; and
saving the trained models according to the provided labels of the genus and species data files.

10. The classification method of claim 8, further comprising:
receiving a genomic sequence file at the input device;
preprocessing the genomic sequence file;
analyzing the genomic sequence file with the genus classifier model to identify a corresponding genus output; and
analyzing the genomic sequence file with the species classifier model associated with the genus output to identify a corresponding species output.

11. The classification method of claim 10, wherein the genomic sequence file is in the form of FASTA or FASTQ file formats.

12. The classification method of claim 10, wherein the preprocessed regions are used as biological markers to identify similarities with known classes of genus or species outputs.

13. The classification method of claim 10, wherein the genomic sequence file is normalized by using padding to ensure consistency in input format and length.

14. The classification method of claim 8, wherein the processing component comprises:
a central processing unit comprising at least one processor configured to perform systematic operations upon data.
